(19) United States
(12) Patent Application Publication
(10) Pub. No.: US 2004/0063618 A1
(43) Pub. Date: Apr. 1, 2004

(54) Peptide nucleic acids having improved uptake and tissue distribution

(76) Inventor: Muthiah Manoharan, Carlsbad, CA (US)

Correspondence Address: WOODCOCK WASHBURN LLP, ONE LIBERTY PLACE - 46TH FLOOR, PHILADELPHIA, PA, 19103, US

(21) Appl. No.: 10/261,382

(22) Filed: Sep. 30, 2002

(51) Int. Cl. A61K 38/16; A61K 48/00; C12N 15/85; C07K 14/00

(52) U.S. Cl. 514/8; 435/455; 530/350; 530/395

(57) ABSTRACT

Disclosed are compositions and methods for enhancing in vivo uptake and tissue distribution in animals of peptide nucleic acids. The peptide nucleic acids include cationic conjugates attached thereto. The cationic conjugated peptide nucleic acids exhibit enhanced uptake and tissue distribution.

### BACKGROUND OF THE INVENTION

The present invention provides compositions and methods for enhancing in vivo uptake and tissue distribution of peptide nucleic acids in animals. In particular, this invention related to peptide nucleic acids having cationic conjugates attached thereto and to method of using these cationic conjugated peptide nucleic acids for enhanced uptake and tissue distribution.

Peptide nucleic acids, alternately referenced as PNAs, are known to be useful as oligonucleotide mimetics. In PNAs, both the sugar and the internucleoside linkage, i.e., the backbone, of the nucleotide units of oligonucleotides are replaced with novel groups. The sugar-backbone of an oligonucleotide is replaced with an amide containing backbone, in particular an aminoethylglycine backbone. The nucleobases are retained and are bound directly or indirectly to aza nitrogen atoms of the amide portion of the backbone. The base units, i.e., nucleobases, are maintained for hybridization with an appropriate nucleic acid target compound.

PNAs have been shown to have excellent hybridization properties as well as other properties useful for diagnostics, therapeutics and as research reagents. They are particularly useful as antisense reagents. Other uses include monitoring telomere length, screening for genetic mutations and for affinity capture of nucleic acids. As antisense reagents they can be used for transcriptional and translational blocking of genes and to effect alternate splicing. Further they can be used to bind to double stranded nucleic acids. Each of these uses are known and have been published in either the scientific or patent literature.

The synthesis of and use of PNAs has been extensively described. Representative United States patents that teach the preparation of and use of PNA compounds include, but are not limited to, U.S. Pat. Nos. 5,539,082; 5,5539,083; 5,641,625; 5,714,331; 5,719,262; 5,766,855; 5,773,571; 5,786,461; 5,831,014; 5,864,010; 5,986,053; 6,201,103; 6,204,326; 6,210,892; 6,228,982; 6,350,853; 6,414,112; 6,441,130; and 6,451,968, each of which is herein incorporated by reference. Additionally PNA compounds are described in numerous published PCT patent applications including WO 92/20702. Further teaching of PNA compounds can be found in scientific publications. The first such publication was Nielsen et al., Science, 1991, 254, 1497-1500.

Depending on its sequences, the solubility of PNAs can differ and, as such, some PNA sequences are not soluble as might be desirable for a particular use. It was suggested in Karras, et. al., Biochemistry, 2001, 40, 7853-7859, that PNAs could mediate splicing activity in cells. They compared a PNA 15mer (a PNA having 15 mononeric units) to the same PNA have a single lysine amino acid jointed to its C terminus. They suggested that the attached, i.e., conjugated, lysine residue might improve the cellular uptake. However, they concluded that their present data “do not show a clear difference in activity between the PNA 15mer with and without a C-terminal lysine.”

In published application, US-2002-0049173-A1, published Apr. 25, 2002, it was suggested that antisense compounds might have one or more cationic tails, preferable positively-charged amino acids such as lysine or arginine, conjugated thereto. It was further suggested that one or more lysine or arginine residues might be conjugated to the C-terminal end of a PNA compound. No discrimination was made between the effects resulting from the conjugation of one lysine or arginine verses more than one of these lysine or arginine residues.

### BRIEF DESCRIPTION OF THE INVENTION

Contrary to the assertions above, it has now been discovered that the properties lysine, hisidine, omithine or arginine conjugates impart to PNA compounds (also described as PNA oligomers, peptide nucleic acid compounds and PNA oligomers) is dependent on the exact number of lysine, hisidines, ornithine or arginines residues conjugated to the PNA compound. It has been discovered that PNA compounds having multiple cationic amino acids conjugated thereto exhibited enhanced intracellular accumulation, enhanced antisense efficiency and tissue distribution. This enhanced uptake, antisense efficiency and tissue distribution of multiple cationic charged PNA compounds was surprisingly greater than that of single charged PNA compounds, neutral PNA compounds and neutral or anionic charged antisense compounds.

It is therefore an object of this invention to provide PNA compound bearing conjugate groups that have multiple cationic charges. It is a further object of this invention to provide methods of using these PNA compounds having cationic conjugate groups for modulating in vivo uptake of PNA compounds.

Therefore, one aspect of the invention is directed to providing a method of modulating in vivo uptake of a peptide nucleic acid compound that includes modifying the peptide nucleic acid molecule with a positively charged conjugate and where the positively charged conjugate has at least three positively charged amino acid units.

A further aspect of the invention includes selecting the positively charged conjugate to include at least three lysine, histidine, ornithine or arginine amino acid units. In an additional aspect of this invention the L-form of the lysine, histidine, ornithine or arginine amino acid units are selectted.

An additional aspect of the invention includes selecting the positively charged conjugate to include at least at least four lysine, hisidine, ornithine or arginine amino acid units. Particularly preferred positively charged conjugates are positively charged conjugates that include at least four lysine, or arginine amino acid units.

The invention further includes a method of modulating tissue distribution of a peptide nucleic acid compound that includes modifying the peptide nucleic acid molecule with a positively charged conjugate where the positively charged conjugate has at least three positively charged amino acid units.

The invention further includes a method of increasing cellular uptake of a peptide nucleic acid compound that includes modifying the peptide nucleic acid molecule with a positively charged conjugate where the positively charged conjugate has at least three positively charged amino acid units.

The invention further includes a method of modulating tissue distribution of a peptide nucleic acid compound in an animal that includes modifying the peptide nucleic acid molecule with a positively charged conjugate where the positively charged conjugate has at least three positively charged amino acid units and delivering the modified peptide nucleic acid compound to said animal.

As used in this invention peptide nucleic acid conjugates include compounds of the formula:

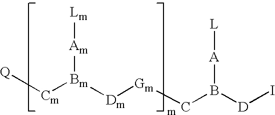

wherein:

m is an integer from 1 to about 50;

L and Lₘ independently are R¹²(R¹³)ₐ; wherein:

R¹² is hydrogen, hydroxy, (C₁-C₄)alkanoyl, a naturally occurring nucleobase, a non-naturally occurring nucleobase, an aromatic moiety, a DNA intercalator, a nucleobase-binding group, a heterocyclic moiety, a reporter ligand, a conjugate or a cationic conjugate;

provided that at least one of R¹² is a naturally occurring nucleobase, a non-naturally occurring nucleobase, a DNA intercalator, or a nucleobase-binding group;

R¹³ is a conjugate; and

a is 0 or 1;

C and Cₘ independently are (CR⁶R⁷)_{y}; wherein:

R⁶ and R⁷ independently are hydrogen, a side chain of a naturally occurring alpha amino acid, (C₂-C₆) alkyl, aryl, aralkyl, heteroaryl, hydroxy, (C₁-C₆) alkoxy, (C₁-C₆) alkylthio, a conjugate, a cationic conjugate, NR³R⁴, SR⁵ or R⁶ and R⁷ taken together complete an alicyclic or heterocyclic system;

wherein R⁵ is hydrogen, a conjugate, or a cationic conjugate,(C₁-C₆)alkyl, hydroxy-, alkoxy-, or alkylthio-substituted (C₁-C₆)alkyl; and

R³ and R⁴ independently are hydrogen, a conjugate, or a cationic conjugate, (C₁-C₄)alkyl, hydroxy- or alkoxy- or alkylthio-substituted (C₁-C₄)alkyl, hydroxy, alkoxy, alkylthio or amino;

D and Dₘ independently are (CR⁶R⁷)_{z};

each of y and z is zero or an integer from 1 to 10, wherein the sum y+z is greater than 2 but not more than 10;

Gₘ is independently —NR³CO—, —NR³CS—, —NR³SO—, or —NR³SO₂— in either orientation;

each pair of A-Aₘ and B-Bₘ are selected such that:

(a) A or Aₘ is a group of formula (IIa), (IIb) or (IIc) and B or Bₘ is N or R³N⁺; or

(b) A or Aₘ is a group of formula (IId) and B or Bₘ is CH;

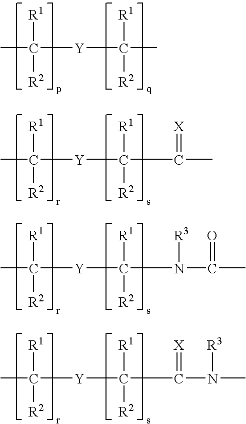

wherein:

X is O, S, Se, NR³, CH₂ or C(CH₃)₂;

Y is a single bond, O, S or NR⁴;

each of p and q is zero or an integer from 1 to 5;

each of r and s is zero or an integer from 1 to 5;

R¹ and R² independently are hydrogen, (C₁-C₄)alkyl, hydroxy-substituted (C₁-C₄)alkyl, alkoxy-substituted (C₁-C₄)alkyl, alkylthio-substituted (C₁-C₄)alkyl, hydroxy, alkoxy, alkylthio, amino, or halogen;

I is —NR⁸R⁹ or —NR¹⁰C(O)R¹¹; wherein:

R⁸, R⁹, R¹⁰ and R¹¹ independently are hydrogen, alkyl, an amino protecting group, a reporter ligand, an intercalator, a chelator, a peptide, a protein, a carbohydrate, a lipid, a steroid, a nucleoside, a nucleotide, a nucleotide diphosphate, a nucleotide triphosphate, an oligonucleotide, an oligonucleoside, a soluble polymer, a non-soluble polymer, a conjugate or a cationic conjugate;

Q is —CO₂H, —CO₂R⁸, —CO₂R⁹, —CONR⁸R⁹, —SO₃H, —SO₂NR¹⁰R¹¹ or an activated derivative of —CO₂H or —SO₃H;

wherein at least one of said R³, R⁴, R⁵, R⁶, R⁷, R⁸, R⁹, R¹⁰, R¹¹, R¹² is said cationic conjugate;

wherein said cationic conjugate includes at least three positively charged amino acid units; and

wherein said cationic conjugate optionally includes a linking moiety.

Preferred PNA compounds of the invention are compound of the above formula wherein one of R⁸, R⁹, R¹⁰ and R¹¹ is said cationic conjugate. Other preferred PNA compounds of the invention are compounds of the above formula wherein one of R³, R⁴, R⁵, R⁶ and R⁷.

Preferred PNA compounds of the invention are compounds of the above formula wherein the cationic conjugate is a conjugate having at least three lysine, hisidine, omithine or arginine amino acid units. Particularly preferred PNA compounds of the above formula are compounds having at least four lysine, hisidine, ornithine or arginine amino acid units.

The cationic conjugate of the invention include cationic conjugates having only a single type of amino acid unit, e.g., four lysine units or four arginine units. The cationic conjugates the invention further include having multiple types of amino acid units, e.g., mixtures of lysine, hisidine, omithine or arginine units. Thus a cationic conjugate of the invention could include having four lysine units, four hisidine units, four arginine units, mixtures of lysine and hisidine, mixtures of lysine and ornithine, mixtures of lysine and arginine, mixtures of hisidine and omithine, mixtures of hisidine and arginine, mixtures of ornithine and arginine, mixtures of three of lysine, hisidine, ornithine and arginine or mixtures of all four of lysine, hisidine, omithine and arginine.

### DETAILED DESCRIPTION OF THE INVENTION

As used in this invention peptide nucleic acids are compounds composed of a neutral backbone having nucleobases attached there to via a tether or linking group. These peptide nucleic acids can also be described as PNA compounds, PNA oligomers, peptide nucleic acid compounds or PNA oligomers. The peptide nucleic acids of the invention are compounds of the formula:

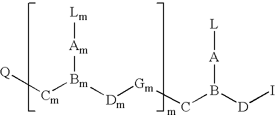

wherein:

m is an integer from 1 to about 50;

L and Lₘ independently are R¹²(R¹³)ₐ; wherein:

R¹² is hydrogen, hydroxy, (C₁-C₄)alkanoyl, a naturally occurring nucleobase, a non-naturally occurring nucleobase, an aromatic moiety, a DNA intercalator, a nucleobase-binding group, a heterocyclic moiety, a reporter ligand, a conjugate or a cationic conjugate;

provided that at least one of R¹² is a naturally occurring nucleobase, a non-naturally occurring nucleobase, a DNA intercalator, or a nucleobase-binding group;

R¹³ is a conjugate; and

a is 0 or 1;

C and Cₘ independently are (CR⁶R⁷)_{y}; wherein:

R⁶ and R⁷ independently are hydrogen, a side chain of a naturally occurring alpha amino acid, (C₂-C₆) alkyl, aryl, aralkyl, heteroaryl, hydroxy, (C₁-C₆) alkoxy, (C₁-C₆) alkylthio, a conjugate, a cationic conjugate, NR³R⁴, SR⁵ or R⁶ and R⁷ taken together complete an alicyclic or heterocyclic system;

wherein R⁵ is hydrogen, a conjugate, or a cationic conjugate,(C₁-C₆)alkyl, hydroxy-, alkoxy-, or alkylthio-substituted (C₁-C₆)alkyl; and

R³ and R⁴ independently are hydrogen, a conjugate, or a cationic conjugate, (C₁-C₄)alkyl, hydroxy- or alkoxy- or alkylthio-substituted (C₁-C₄)alkyl, hydroxy, alkoxy, alkylthio or amino;

D and Dₘ independently are (CR⁶R⁷)_{z};

each of y and z is zero or an integer from 1 to 10, wherein the sum y+z is greater than 2 but not more than 10;

Gₘ is independently —NR³CO—, —NR³CS—, —NR³SO-—or —NR³SO₂— in either orientation;

each pair of A-Aₘ and B-Bₘ are selected such that:

(a) A or Aₘ is a group of formula (IIa), (IIb) or (IIc) and B or Bₘ is N or R³N⁺; or

(b) A or Aₘ is a group of formula (IId) and B or Bₘ is CH;

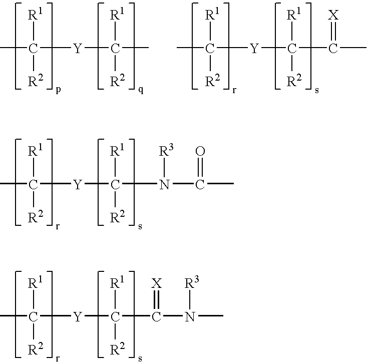

wherein:

X is O, S, Se, NR³, CH₂ or C(CH₃)₂;

Y is a single bond, 0, S or NR⁴;

each of p and q is zero or an integer from 1 to 5;

each of r and s is zero or an integer from 1 to 5;

R¹ and R² independently are hydrogen, (C₁-C₄)alkyl, hydroxy-substituted (C₁-C₄)alkyl, alkoxy-substituted (C₁-C₄)alkyl, alkylthio-substituted (C₁-C₄)alkyl, hydroxy, alkoxy, alkylthio, amino, or halogen;

I is —NR⁸R⁹ or —NR¹⁰C(O)R¹¹; wherein:

R⁸, R⁹, R¹⁰ and R¹¹ independently are hydrogen, alkyl, an amino protecting group, a reporter ligand, an intercalator, a chelator, a peptide, a protein, a carbohydrate, a lipid, a steroid, a nucleoside, a nucleotide, a nucleotide diphosphate, a nucleotide triphothate, an oligonucleotide, an oligonucleoside, a soluble polymer, a non-soluble polymer, a conjugate or a cationic conjugate;

Q is —CO₂H, —CO₂R⁸, —CO₂R⁹, —CONR⁸R⁹, —SO₃H, —SO₂NR¹⁰R¹¹ or an activated derivative of —CO₂H or —SO₃H;

wherein at least one of said R³, R⁴, R⁵, R⁶, R⁷, R⁸, R⁹, R¹⁰, R¹¹, R¹² is said cationic conjugate;

wherein said cationic conjugate includes at least three positively charged amino acid units; and

wherein said cationic conjugate optionally includes a linking moiety.

In preferred PNA compounds of the invention, one of R⁸, R⁹, R¹⁰ and R¹¹ is selected as the cationic conjugate.

The cationic conjugate optionally can include a linking moiety. Optionally the cationic conjugate can include neutral amino acids interspaced between or concatenated with the cationic amino acids. Neutral amino acid useful for this purpose include alanine, leucine, phenylalanine, tryptophan, isoleucine, valine, tyrosine and proline. For example, a cationic conjugate of the invention might include four lysine units (“K” amino acid units) and six phenylalaine units (“F” amino acid units) assembled as KFFKFFKFFK.

Preferred PNA compounds are compounds of the above formula wherein the cationic conjugate is a conjugate having at least three lysine, hisidine, omithine or arginine amino acid units. Particularly preferred PNA compounds of the above formula are compounds having at least four lysine, hisidine, ornithine or arginine amino acid units.

The cationic conjugate of the invention include cationic conjugates having only a single type of amino acid unit, e.g., four lysine units or four arginine units. The cationic conjugates the invention further include having multiple types of amino acid units, e.g., mixtures of lysine, hisidine, ornithine or arginine units. Thus a cationic conjugate of the invention could include having four lysine units, four hisidine units, four arginine units, mixtures of lysine and hisidine, mixtures of lysine and ornithine, mixtures of lysine and arginine, mixtures of hisidine and ornithine, mixtures of hisidine and arginine, mixtures of ornithine and arginine, mixtures of three of lysine, hisidine, ornithine and arginine or mixtures of all four of lysine, hisidine, ornithine and arginine. Thus one or more types of cationic amino acid units can be assembled into a cationic conjugate of at least three cationic amino acid units in length, preferable four amino acid units in length. A particularly preferred amino acid for use as the cationic conjugate is lysine. A further preferred amino acid for use as the cationic conjugate is arginine.

Other preferred PNA compounds of the invention are illustrated in U.S. Pat. No. 6,395,474, therein incorporated by reference. Particularly preferred PNA compounds are compounds having an aminoglycine backbone as illustrated in U.S. Pat. No. 6,395,474.

The sequencing of the human genome and parallel analysis of expressed sequence tag (EST) libraries indicates that at least 35% of all genes code for alternatively spliced pre-mRNAs. In some cases a single pre-mRNA may generate multiple splice variants. For example, the slo and dscam genes can generate 500 and 38,000 unique mRNAs, respectively. Alternative splicing is thus a major contributor to the vast diversity of proteomes. Furthermore, considering that approximately 15% of all genetic diseases are the result of mutations that damage proper splicing pathways, modification of inappropriate alternative splicing emerges as an important approach for controlling gene expression with potential therapeutic outcomes. In addition to genetic diseases, antisense-mediated modification of splicing appears particularly attractive for the treatment of cancer, as changes in splicing are frequently observed in cancer cells.

C-to-T mutation at nucleotide 654 of the human β-globin intron-2 (IVS2-654) activates aberrant 5′ and 3′ splice sites that are preferably utilized during splicing, despite the presence of the normal, unaltered sites. The presence of this mutation in human β-globin gene interferes with correct expression of β-globin, causing thalassemia, a blood disorder. Previous reports have shown that antisense oligonucleotides hybridized to the aberrant β-globin 5′ splice site forced the splicing machinery to use the normal splice sites, resulting in correctly spliced β-globin mRNA. When the β-globin intron containing the mutation at position 654 is inserted at nucleotide 105 of EGFP cDNA, the spliced EGFP mRNA retains a portion of the globin intron, preventing correct translation of EGFP. Treatment of the cells expressing the IVS2-654 EGFP construct with active antisense oligonucleotide should restore proper splicing and translation of EGFP, providing a rapid and sensitive positive readout for antisense activity in the nuclei of the treated cells.

As an illustration of this invention, in vivo effects of various modified antisense oligomers on modulation of splicing in organs and tissues in a transgenic mouse that express the coding sequence of EGFP interrupted by a mutant form of the human β-globin 2^{nd} intron, IVS2-654 was used. The oligomers were complementary to the aberrant 5′splice site in the modified EGFP pre-mRNA and were delivered systemically.

A transgenic mouse has been developed that expresses the coding sequence of enhanced green fluorescent protein (EGFP) interrupted by a mutant form of the human β-globin 2^{nd} intron, IVS2-654. Aberrant splicing prevents expression of EGFP in all tissues. However, EGFP production can be restored if splicing is corrected by antisense oligonucleotide treatment. This mouse model was used to inspect the in vivo antisense activity of oligonucleotide analogues including 2′-O-methoxyethyl (MOE) phosphorothioates, morpholino oligomers, and peptide nucleic acids (PNAs).

In antisense-treated EGFP-654 mice, significant antisense activity was seen in a number of tissues including liver, kidney, heart, lung, small intestine and muscle. In brain, skin and stomach no or only marginal levels of activity were detected.

In animals, it has been found that a PNA with a lysine conjugate at the C-terminus exhibited the highest overall antisense activity after systemic injection. For illustrative purposes up-regulation of the EGFP gene was measured. The PNA compounds conjugated with multiple lysine units up-regulated EGFP in several tissues including the liver, kidney and heart. In contrast, the PNA oligomer with only one lysine was completely inactive, underlining the importance of the cationic conjugate for use in vivo.

Comparing the effects of the different chemistries evaluated in this study, the PNA-4K compound had the highest overall activity. In vivo the PNA oligomer with only one lysine (PNA-IK) showed no detectable levels antisense activity in any tissues assayed, even at the high doses used (50 mg/kg). While we do not wish to be bound by theory, these data suggest that the 4 lysine moiety on PNA-4K contributes substantially to the in vivo activity by promoting its uptake into the cells and tissues.

The results from EGFP read-out were confirmed on the mRNA level by utilizing an RT-PCR assay. In tissues with no or only marginal EGFP signal, no increase in correctly spliced mRNA could be detected, whereas tissues with high fluorescence signals showed correspondingly significant shifts in splicing. None of the control oligomers were active in any tissues examined including those where high levels of specific antisense activity were observed.

### EXAMPLE 1

PNA Synthesis

Peptide nucleic acids (PNAs) can be prepared in accordance with any of the various procedures referred to in Peptide Nucleic Acids (PNA): Synthesis, Properties and Potential Applications, Bioorganic & Medicinal Chemistry, 1996,4, 5-23. They may also be prepared in accordance with U.S. Pat. Nos. 5,539,082, 5,700,922, 5,719,262 and 6,395,474, herein incorporated by reference. Using these teachings, PNA oligomers were synthesized in 10 μmol scale on a 433A Applied Biosystems Peptide Synthesizer using commercially available t-butyloxycarbonyl/benzyloxycarbonyl (Boc/Cbz)-protected monomers (Applied Biosystems) and synthesis protocols based on previously published procedures. The coupling efficiency was monitored by qualitative Kaiser test

### EXAMPLE 2

Cationic Conjugated PNA

The C-terminal L-lysines (Lys) were introduced by using a resin pre-loaded with Boc-Lys(2-Cl-Z)-OH. Further Lys residues were introduced during solid-phase synthesis using the protocols for PNA synthesis. After cleavage and deprotection the PNA oligomers were purified by reversed-phase high performance liquid chromatography (RP-HPLC), analyzed by electrospray ionization mass spectrometry, lyophilized and stored at-20° C. Synthesis on a 10 μmol scale yielded >20 mg of PNA oligomer with a purity of >95% after RP-HPLC purification. In a like manner, C-terminal hisidine, ornithine and arginine are introduced using Boc blocked hisitidine, omithine and arginine amino acids precursors. Other blocking groups can also be selected to protect the amino acid units during synthesis of the conjugate groups.

### EXAMPLE 3

Antisense oligonucleotides

Antisense oligomers are prepared using standard protocols. The antisense oligomers were synthesized as 2′-O-methyl (2′-O-Me)phosphorothioate oligonucleotide (PTOs), 2′-O-methoxyethyl (2′-O-MOE) PTOs or morpholino oligomers. 2′-O-Me oligonucleotides were purchased from TRI-Link, Inc. (San Diego, Calif.). 2′-O-MOE-modified oligonucleotides were synthesized on an automated DNA synthesizer (Applied Biosystems, model 380B) using standard phosphoramidite chemistry. The oligonucleotides were analyzed by capillary gel electrophoresis and judged to be at least 90% full-length material. Morpholino oligonucleotides were synthesized as described elsewhere by stirchak, et. al., Nucleic Acid Research, 17, 6129-6141 or Kaiser, et. al., Anal. Biochem., 49, 595-598. Tetra methyl rhodamine (TAMRA), Texas Red and fluorescein (FITC) were used to label 2′-O-Me, 2′-O-MOE and morpholino oligomers, respectively. For certain of the examples of this invention, the PNA and other antisense oligonucleotides were synthesized as 18mers complementary to the β-globin intron 2 at the aberrant 5′ splice site around position 654. Control oligonucleotides were targeted downstream, around position 705.

Compounds as prepared as described in examples 1-3, above, are listed in Table I below and are used in the further examples.

TABLE 1
Sequence and backbone modification of the oligomers synthesized

| | | Target | |
|---|---|---|---|
| Oligomer | Sequence 5′→3′ | site | Backbone |
| SEQ ID No. 1 | GCT ATT ACC TTA ACC CAG | 654 | 2′-O-Me, P = S |
| SEQ ID No. 2 | GCT ATT ACC TTA ACC CAG | 654 | 2′-O-MOE, P = S |
| SEQ ID No. 3 | GCT ATT ACC TTA ACC CAG | 654 | Morpholino |
| SEQ ID No. 4 | H-GCT ATT ACC TTA ACC CAG-Lys-NH₂ | 654 | PNA |
| SEQ ID No. 5 | H-GCT ATT ACC TTA ACC CAG-(Lys)₂-NH₂ | 654 | PNA |
| SEQ ID No. 6 | H-GCT ATT ACC TTA ACC CAG-(Lys)₄-NH₂ | 654 | PNA |
| SEQ ID No. 7 | CCT CTT ACC TCA GTT ACA | 705 | 2′-O-Me, P = S |
| SEQ ID No. 8 | CCT CTT ACC TCA GTT ACA | 705 | 2′-O-MOE, P = S |
| SEQ ID No. 9 | CCT CTT ACC TCA GTT ACA | 705 | Morpholino |
| SEQ ID No. 10 | H-CCT CTT ACC TCA GTT ACA-Lys-NH₂ | 705 | PNA |
| SEQ ID No. 11 | H-CCT CTT ACC TCA GTT ACA-(Lys)₂-NH₂ | 705 | PNA |
| SEQ ID No. 12 | H-CCT CTT ACC TCA GTT ACA-(Lys)₄-NH₂ | 705 | PNA |
| SEQ ID No. 13 | H-GCT ACT ACA TTA AAC CAG-(Lys)₄-NH₂ | 654 | PNA |
| | | 3MM | |
| SEQ ID No. 14 | H-CCA CTT ACC TCA GTT ACA-(Lys)₄-NH₂ | 705u | PNA |

In discussing the above described compounds, the compound of SEQ ID No. 1 will also be referenced as oligomer 1. Reference to the other compounds are made in the same manner, e.g., oligomer 2 is SEQ ID No. 2.

### EXAMPLE 4

Plasmid and Cell Line Construction

Insertion of the mutated human 13-globin intron, IVS2-654 at nucleotide 105 of EGFP cDNA was performed by a modified procedure of Jones and Howard, B. H. (1991), Biotechniques, 10, 62-66. Briefly, vector pEGFP-N1 (Clontech, Palo Alto, Calif.) was linearized by PCR (one cycle at 95° C., 3 min; 30 cycles, 95° C., 1 min; 60° C., 1 min; 72° C., 5 min) with overlapping forward (5′-GGCGATGCCACCTACGGCAAGC-3′). SEQ ID No. 15, and reverse (5′-GAGCGCACCATGTTCTTCAAGG-3′), SEQ ID No. 16, primers. PCR of plasmid IVS2-654 with forward (5′-CGTGTCCGGCGAGGGCGAGGTGAGTCTATGGGACCC-3′), SEQ ID No. 17, and reverse (5′-GCTTGCCGTAGGTGGCATCGCCCTGTGGGAGGAAGATAAG-3′), SEQ ID No. 18, primers under the same conditions produced a linear IVS2-654 intron with the ends homologous to EGFP sequence. Transformation of Max DH5X cells (Life Technologies, Rockville, Md.) with both DNA fragments led to homologous recombination and generation of the plasmid with IVS2-654 inserted in the coding sequence of EGFP.

HeLa S3 cells were transfected with 1 μg of IVS2-654 EGFP plasmid DNA by lipofection as suggested by the manufacturer (4 μl lipofectamine; Life Technologies). Stable cell lines were selected after 7-14 days in culture in minimum essential medium (MEM), supplemented with 5% fetal calf serum, 5% horse serum and 400 μg/ml G418.

### EXAMPLE 5

Oligonucleotide Delivery

HeLa cells expressing the IVS2-654 EGFP construct were maintained at below 80% confluence in S-MEM (Gibco-BRL) supplemented with 5% fetal calf serum, 5% horse serum and antibiotics. For scrape loading, cells were seeded 24 h before treatment in 24-well plates at ˜10⁵ cells per well in 0.5 ml of medium. For free uptake experiments, cells were plated in 96-well plates at 8×10³ cells per well in 150 μl of medium. For monolayers to be scrape-loaded the medium was aspirated and 0.5 ml of growth medium containing antisense oligonucleotides (concentrations as shown in figure legends) was applied. Cells were then scraped off the plate with a cell scraper (Costar, Corning, N.Y.), replated in a fresh 24-well dish and assayed 24 h later. In free uptake experiments, growth medium was removed and replaced with 150 μl of fresh growth medium containing oligonucleotides. Cells were assayed 24 h later or as indicated in the figure legends.

### EXAMPLE 6

RNA Isolation and RT-PCR

Oligonucleotide-treated cells were lyzed in 0.8 ml of TRI-reagent (MRC, Cincinnati, Ohio) and total RNA was isolated. A 100 ng sample of RNA was used in RT-PCR with rTth enzyme (Perkin-Elmer, Branchburg, N.J.) in the presence of 0.2 μCi of [α-³²P]dATP. Both procedures followed the manufacturer's protocols. The reverse transcription reaction was carried out at 70° C. for 15 min followed by PCR: 1 cycle, 95° C., 3 min; 18 cycles, 95° C., 1 min; 65° C., 1 min. For EGFP mRNA amplification forward and reverse primers were 5′-CGTAAACGGCCACAAGTTCAGCG-3′ SEQ ID. No. 19 and 5′-GTGGTGCAGATGAACTTCAGGGTC-3′ SEQ ID No. 20, respectively. The latter primer was used in the reverse transcription step. For β-globin, the forward and reverse primers spanned position 21-43 of exon 2 and position 6-28 of exon 3, respectively, as described in Sierakowska et al., Proc. Natl Acad. Sci. USA, 93, 128401-12844. The PCR products were analyzed by electrophoresis on an 8% non-denaturing polyacrylamide gel. Gels were dried and autoradiographed with Kodak Biomax film at-80° C. Images were digitized by scanning with a Hewlett Packard scanner using Adobe Photoshop software.

### EXAMPLE 7

### Flow Cytometry

Cells were trypsinized in 24- and 96-well plates with 200 and 100 μl 1× trypsin (Sigma, St Louis, Mo.), respectively, for 2 min at 37° C. and resuspended in 1-2 ml of growth media. Approximately 104 cells from each sample were subjected to flow cytometry with a Becton-Dickinson FACScan (San Jose, Calif.) (flow rate=100-200 cells/s). Dead or abnormal cells were omitted by gating of side versus forward scatter and histograms of green fluorescence intensity versus cell number were generated. The total mean fluorescence of the mock-treated controls was set to ˜10¹ and the gate used for analysis of treated cells set to include 2.5% of most brightly fluorescent control cells as background. Consequently, treated samples could be analyzed in terms of a fluorescence index (FI). This number is derived by multiplying the percentage of cells scoring above the background threshold by the mean fluorescence intensity of that sub-population. Experimental conditions were established so that mock and untreated samples had a Fl of 1.

### EXAMPLE 8

Fluorescence Microscopy

Cell culture medium was replaced with HBSS and bright field and UV images were taken using an inverted Olympus microscope (10× objective). Images were digitized using the Olympus digital imaging system and stored on a Power PC running Scion Image 1.62a software.

### EXAMPLE 9

Confocal Microscopy

HeLa EGFP-654 or HeLa cells not expressing EGFP-654 were cultured on 8-well slide wells at ˜2×10⁵ cells per well. Scrape loading was performed in a 24-well plate as described above, except that the cells were transferred to a new slide not a 24-well plate. For free uptake and cationic lipid transfections, treatment with the oligomer was performed in the slide well. Twenty-four hours after treatment, the cells were rinsed twice with PBS and fixed on the slide with 2% paraformaldehyde. Glass coverslips were mounted with Vecta-shield and sealed with nylon epoxy. Confocal images were taken within 48 h with an Olympus confocal microscope. For double staining, sequential scanning of each fluorophor was performed to prevent cross detection. Images were saved as TIFs and, when necessary, merged in Adobe Photoshop.

### EXAMPLE 10

Toxicity Assay

Approximately 10⁴ cells/well were seeded in 96-well plates for 24 h. Media was then replaced with 100 μl media containing increasing amounts of free oligonucleotide. After 24 h, MTS (Promega, Madison, Wis.) was added directly to the culture wells as indicated by the manufacturer and the plates were incubated at 37° C. for 2 h. Absorbance at 490 nm was measured and compared with that of mock-treated samples.

### EXAMPLE 11

IVS2-654 EGFP Reporter Cell Line A

Transfection of the IVS2-654 EGFP HeLa cell line with 2′-O-Me-PTO oligonucleotide targeted to the aberrant 5′ splice site (ON-654,oligomer 1 in Table 1) and complexed with lipofectamine, a cationic lipid, resulted in up-regulation of the EGFP-IVS2-654 gene, detected as bright fluorescence on a gel. Fluorescent activated cell sorting (FACS) analysis of treated cells showed an increase in the population of cells with fluorescence intensity ˜15-fold higher than the baseline. The fluorescence did not increase in mock-treated cells or cells treated with a control oligonucleotide ON-705 (oligomer 7, Table 1). Oligomer 7 hybridizes to a region of the intron 51 nt downstream from the IVS2-654 mutation and repairs splicing in another thalassemic mutant, IVS2-705. It is also partially complementary to the IVS2-654 splice site, with only six mismatches if G-U or G-T base pairing is taken into account. Thus, oligomer 7 provides a stringent control for sequence specificity of the antisense effects of ON-654. The use of oligonucleotides against constructs containing the IVS2-654 sequence and evidence of sequence specificity and antisense mechanism of action has also been reported previously (see Schmajuk, et. al., (1999), J. Biol. Chem., 274, 21783-21789; Mercatante, et. al., (2000), Pharmacol. Ther., 85, 237-243; and Lacerra, et. al., (2000) Proc. Natl Acad. Sci. USA, 97, 9591-9596).

To confirm that the induced, green fluorescence was due to correction in splicing of the EGFP pre-mRNA, total cellular RNA was analyzed by RT-PCR. In cells treated with oligomer 1, a shorter band representing correctly spliced EGFP mRNA appeared in addition to a longer product of aberrant splicing; maximal correction occurred at 0.1 μM oligonucleotide. Treatment of the cells with the control oligomer 7 had no effect. As, in this experiment, the concentration of lipofectamine was held constant while the oligonucleotide concentration was increased, the lower activity of oligomer 1 at 0.3 μM is due to inappropriate lipofectamine-ligonucleotide ratio. The results indicate that in a sub-population of treated cells, oligomer 1 crossed the cell membrane, entered the nucleus and in a sequence-specific manner shifted splicing from aberrant to correct in the EGFP system. Thus, the RT-PCR analysis validated the use of fluorescence assay and confirmed that the oligomers acted by affecting splice site choice. Similar results were obtained with the 2′-O-MOE derivative, oligomer 2 and its control oligomer 8.

### EXAMPLE 12

Free Uptake of 2′-O-Me, 2′-O-MOE, Morpholino and PNA Oligomers

To elucidate the influence of the backbone modification on the cellular uptake and antisense properties of different oligonucleotide analogs, the latter were tested in the EGFP assay. Negatively charged (2′-O-Me and 2′-O-MOE) oligomers and neutral or cationic morpholino and PNA oligomers targeted to the 654 splice site were evaluated in cells treated in the absence of transfection reagents. Results were judged as a FI (fluorescent index). This index takes into account the percentage of fluorescent cells in the sample and the intensity of their fluorescence. For example, for 3 μM morpholino and PNA analogs (oligomers 3 and 4), the FI increased from a background of 1 to ˜65 and 80, respectively. The percentage of cells exhibiting fluorescence above background increased to 55 and 70% of the cell population. In contrast, with the same concentration of negatively charged 2′-O-Me and 2′-O-MOE oligonucleotides (oligomers 1 and 2), FIs of only 5 and 20 were observed, respectively; the percentage of cells that scored above background was only 8% for 2′-O-Me and 19% for 2′-O-MOE. Non-linear regression analysis of the dose response data revealed a theoretical limit of the FI specific for each backbone and delivery method. This allowed characterization of each oligomer/delivery combination in terms of an EC₅₀ and a maximal FI (FIₘₐₓ).

The effects of all oligonucleotide analogs are due to hybridization of the antisense oligomer to the target site on pre-mRNA, as mock-treated cells and cells treated with control oligomers targeted against the 705 site showed only background fluorescence. The sequence specificity was further confirmed by the fact that the oligomers targeted to the 654 site were inactive against cells expressing an EGFP construct with an aberrant 5′ splice site located at nucleotide 705 of the intron (data not shown). While not wanting to be bound by theory, it is presently believed that overall, the results suggest that neutral and cationic morpholino and PNA oligomers more readily cross the cell membrane barrier and gain access to the nucleus than their anionic counterparts (2′-O-MOE and 2′-O-Me).

To assess the contribution of uptake through the cell membrane on the antisense efficacy of the four oligonucleotide-analogs, the oligomers were delivered to cells by scrape loading. Scrape loading facilitates entry of large molecules into cells as a result of mechanical damage to the cell membrane. By this method, PNA, morpholino and 2-O-MOE oligomers 4, 3 and 2, respectively, led to a dose-dependent and very similar increase in the population of fluorescent cells, while the effects were less pronounced for the 2′O-Me oligomer 1. Again while not wanting to be bound by theory, it is presently believed that his suggests that the observed differences in the antisense efficacy of oligomers in the absence of transfection reagents are predominantly a function of their ability to cross the cell membrane.

### EXAMPLE 13

Antisense Efficacy of PNA is Influenced by the Number of Attached Lysine Residues

To further examine the effects of the backbones on the antisense properties of the oligomers, antisense PNAs modified with one, two and four positively charged Lys residues at the C-terminus (PNA-1, -2 and -4; oligomers 4, 5 and 6 in Table 1) were compared. Significant, dose-dependent increases in fluorescence of the cells treated with the Lys-modified PNAs were apparent. Quantitative analysis of FACS data from several experiments clearly demonstrated that the PNA containing four Lys residues (PNA-4, oligomer 6) was the most effective in generating EGFP fluorescence in treated cells; its EC₅₀ (2.1 μM) was almost 2.5 times lower than that of PNA-1 (4.7 μM, oligomer 4). The FIₘₐₓ was comparable with each of the modified PNAs suggesting that at high concentrations all three derivatives are highly effective. For all three derivatives ˜70% of the cell population became fluorescent suggesting that the Lys conjugate increased the actual concentration of the oligonucleotide within the cells rather than the number of transfected cells.

In contrast, no difference was observed in the EC₅₀, FIₘₐₓ or the FIs in cells scrape loaded with PNA-1, -2 and -4 at any tested concentration. These results indicate that the Lys residues attached to the C-terminus of PNA oligomers did not increase their affinity to the target sequence nor influence the nuclear translocation process. Rather, the observed Lys-dependent enhancement of the antisense efficacy in free uptake experiments must have resulted from improved transport of the PNA molecules through the cell membrane or from an enhanced release from the endosomes. Although direct measurement of nuclear accumulation was not possible because of the lack fluorescent labeled PNAs, the above data along with data from the labeled oligomers indicate that the added Lys increased cellular uptake and thus nuclear accumulation of the free PNA derivatives.

### EXAMPLE 14

Uptake of PNA-4

To gain an understanding of how the (Lys)₄ conjugate increased the antisense efficacy of PNA oligomers, cells were incubated with PNA-4 (oligomer 6) for 3 h at a 10 μM concentration and at either 4 or 37° C. For comparison, morpholino (oligomer 3) and 2′-O-MOE (oligomer 2) derivatives were also tested. After treatment, the oligomers were removed by rinsing the cells with culture media and the cells were allowed to recover at 37° C. for 20 h. Incubation at 4° C. lowered the overall intensity of fluorescence of the cells treated with any nucleotide; however, only in cultures treated with PNA-4 did the number of fluorescent cells remain the same, regardless of the incubation temperature. This result suggest that the mechanism of uptake of positively charged PNA derivative is different from those of neutral morpholino and negative 2′-O-MOE analogs. A time course experiment with oligomers 1, 2, 3 and 6 at 1 μM concentration was carried out. All oligomers exhibited a time-dependent increase in EGFP fluorescence, but the rate for the positively charged PNA-4 was higher than those observed for the other oligomers with neutral or anionic backbones. Between 12 and 48 h of incubation with PNA-4 (oligomer 6) the FI increased 20-fold, while only a 10-fold increase was observed for both the morpholino and 2′-O-MOE derivatives 3 and 2, respectively. It was noted that the FI value for oligomer 3 is approximately nine times higher that the FI value of oligomer 2 at 12 h. This further suggests that the uptake properties of PNA-4 are unique compared with those shared by neutral morpholino and negatively charged PTOs.

### EXAMPLE 15

Positively Charged Oligomers are Not Toxic and Sequence Specific

Toxicity of oligonucleotide analogs, especially of cationic derivatives, in free uptake experiments was considered since high concentrations of up to 10 μM were used. However, the growth rates of mock-treated cells and cells treated with the antisense oligomers were comparable, indicating that these compounds do not cause cytotoxicity at the concentrations tested. In addition, toxicity of PNA-4 was analyzed by MTS assay at a 10 μM concentration. No toxicity was observed despite the presence of the (Lys)₄ conjugate at the C-terminus of the oligomer. Normal cell growth rate and lack of toxicity suggest that PNA-4 and the other tested oligomers do not significantly interfere with splicing of non-target RNAs or with other cellular processes, further confirming the sequence specificity of the observed antisense effects. The sequence specificity of the PNA-4 oligomer was tested using a three-mismatch control (oligomer 13) and an oligomer directed to a region of IVS-250 bases downstream (oligomer 14). These oligomers had negligible effects on splicing at any concentration tested.

### EXAMPLE 16

Application of the EGFP-654 Reporter Assay

The PNA-4 (oligomer 6) and morpholino (oligomer 3) oligomers were used in the previously developed cellular model of B-thalassemia to test if the results obtained in the EGFP-654 assay are relevant to models of clinical disease. Treatment of the cells with either oligonucleotide in the absence of transfection reagents led to restoration of correct splicing of the IVS2-654 human B-globin pre-mRNA. Importantly, analysis of RT-PCR results indicated that oligomer 6 was approximately four times more effective than oligomer 3 at correcting pre-mRNA splicing. These results are in qualitative and quantitative agreement with those obtained in the EGFP based assay. This confirms the utility of the latter system in predicting effectiveness of different oligonucleotide chemistries in modification of splicing pathways.

### EXAMPLE 17

EGFP-654 Transgenic Mouse.

An EGFP-654 based assay for in vivo application was adapted, generating a mouse model in which the EGFP-654 transgene, cloned under chicken β-actin promoter is expressed uniformly throughout the body. As a result, the functional effects of the same oligonucleotide can be monitored in almost every tissue. This is in contrast to oligonucleotides targeted to genes whose expression is restricted to or is phenotypically relevant in only certain. As a positive control for EGFP production, a mouse line expressing the wild type β-globin intron (EGFP-WT) was generated. RT-PCR of total RNA isolated from various tissues showed expression of EGFP-WT and EGFP-654 in all tissues surveyed for both mouse lines. For EGFP-WT, a PCR product band for the correctly spliced message (87 base pairs) was observed, while the corresponding mRNA in EGFP-654 mouse line was almost exclusively aberrantly spliced (160 base pairs). In some organs, especially the liver, very low levels of correctly spliced message (87 base pairs) were detectable, indicative of tissue-specific alternative splicing. The RT-PCR reaction was carried out at 18 cycles and with less than 200 ng of RNA per sample, i.e. conditions in which the amplification was in the linear range. The RT-PCR results were confirmed by examination of 10 μm frozen sections by fluorescence microscopy. Bright green fluorescence was detected in every tissue of EGFP-WT mouse, whereas no significant signal was detected in similar samples from the EGFP-654 mouse. These results indicate that the level of either aberrantly or correctly spliced mRNA is fairly uniform in all tissues for each mouse line. However, since the actual target of antisense oligonucleotides that shift splicing is pre-mRNA, pre-mRNA levels in EGFP-WT and EGFP-654 mice were also examined by performing RT-PCR with an intron specific primer. In the EGFP-654 mice, the pre-mRNA was readily detectable in all tissues although smaller amounts were found in the bone marrow, skin and brain. In contrast, very little pre-mRNA from the EGFP-WT mouse was detected under the same RT-PCR conditions. Assuming that the rate of transcription driven by the same chicken β-actin promoter was similar for EGFP-WT and EGFP-654 genes, these results suggest that the wild-type intron was spliced very rapidly, resulting in low steady-state levels of pre-mRNA. On the other hand, if splicing of the IVS2-654 intron were much less efficient, pre-mRNA would accumulate. Importantly, however, the results indicate that for EGFP-654 the target pre-mRNA was present in all examined tissues providing a target for antisense oligonucleotides that are capable of blocking aberrant splice sites. The level of translated EGFP should therefore be proportional to the potency of the antisense oligomers and their concentration at the site of action.

### EXAMPLE 18

Ex-Vivo Treatment of Primary Fibroblasts and Hepatocytes from EGFP-654 Mouse.

To confirm that cells derived from the EGFP-654 mice respond to antisense treatment in a known manner, primary fibroblasts and hepatocytes were isolated and treated with the 18-mer 2′-O-methyl (2′-O-Me) oligoribonucleoside phosphorothioate, oligomer 1, delivered in a complex with cationic lipids. Mock-treated fibroblasts exhibited little or no fluorescence whereas treatment with the oligonucleotide resulted dose-dependent increase in EGFP production. A control oligonucleotide, oligomer 7, targeted to a region downstream of the 654 mutation had no effect, indicating that the effects were sequence specific. In cultured primary hepatocytes, although low levels of autofluorescence were detected in mock- or control-treated samples, robust EGFP derived fluorescence was seen exclusively in oligonucleotide-treated samples with maximum signal detected at 0.1 μM. To confirm that the up-regulation of EGFP was due to a shift in splicing of EGFP-654, total RNA from treated samples was subjected to RT-PCR. For mock- and control-treated samples, only one band corresponding to an aberrant splice product was detected, with little or no correctly spliced message present. Cells treated with the antisense oligonucleotide, oligomer 1, in contrast, showed a significant correction of splicing, with optimal levels occurring at either 0.1 μM or 0.3 μM concentration for fibroblasts and hepatocytes, respectively. In both cases the percentage of correct EGFP mRNA was approximately 40%. These results are in agreement with the corresponding fluorescence images and confirm that in cells from the EGFP-654 transgenic mouse correct splicing of pre-mRNA can be restored by antisense oligonucleotides.

### EXAMPLE 19

Modification of Splicing by Systemic Delivery of Antisense Oligonucleotides to EGFP-654 Mouse.

In this example the pharmacology of oligonucleotides in vivo was examined and correlated to antisense activity observed in the above described in vitro examples. EGFP-654 mice were treated with 50 mg/kg intraperitoneal (IP) injections of the oligomers once a day for 1 or 4 days. This schedule was previously shown to be effective with 2′-O-MOE/2′-deoxy-phosphorothioate chimeras used for down-regulation of fas-ligand in murine liver. The experiments included 2′-O-MOE, morpholino, PNA-1K and PNA-4K 18-mers (oligomers 2-5, Table 1) targeted to the aberrant 5′ splice site of EGFP pre-mRNA. Mock-treated animals and animals treated with mismatched oligomers served as a negative control groups. Animals were sacrificed one day after the final treatment and examined for the presence of EGFP in 10 μm frozen sections from various organs. Tissues from a mock-treated EGFP-654 mouse show minimal fluorescence background likely due to tissue auto-fluorescence or in liver to a small amount of correct splicing of EGFP-654 pre-mRNA. In antisense-treated mice, background or barely detectable fluorescence was seen in brain, skin and stomach. Significant antisense activity was detected in a number of tissues including liver, kidney, heart, lung, small intestine and muscle. Overall, oligomer 4 (PNA-4K) showed the highest potency, while in most of the tissues the morpholino oligomer 3 was the least effective. The effects of the 2′-O-MOE oligomer 2 were somewhat lower than those of PNA-4K, except in the small intestine, where the 2′-O-MOE was more effective. The high fluorescence intensity observed in the small intestine of mice treated with 2′-O-MOE and PNA-4K oligomers probably reflects a high local concentration of the IP injected compounds, although under the same conditions effects of morpholino were barely detectable after one day of treatment. Fluorescent EGFP was produced in several structures, including the villi, the lamina propria and the smooth muscle lining of the small intestine, suggesting that the oligomers were taken up from the solution and penetrated from the outside to the internal tissue layers. The fact that other organs also exhibited production of EGFP indicated that the oligomers were distributed by the blood stream throughout the animal.

Treatment with PNA-4K and 2′-O-MOE oligomers elicited high EGFP levels in parenchymal liver cells, in the cortex of the kidney, and in the cardiac muscle. Strong EGFP signal was also visible in the lung. Interestingly, treatment with the morpholino oligomers, although less effective in other tissues, generated a bright signal in the lining of a large terminal bronchiole. This could indicate a rapid clearance of morpholino oligomers from circulation at least partly by respiration, leading to accumulation, and therefore specific antisense effects in parts of the lung. All oligomers exhibited antisense activity in the skeletal muscle of the thigh. The thigh is the only tissue where the morpholino oligomer appears to be more effective than the 2′-O-MOE oligomer, although a 4-day treatment schedule was needed to exhibit this effect. Approximately equal, but weak, fluorescent EGFP signal was also detected in pancreatic cells for 2 ′-O-MOE, PNA-4K and morpholino oligomers. Weak, but significant, EGFP signal was detected in the red pulp of the spleen and in the cortex of the thymus, but only after 4 daily injections of 2′-O-MOE or PNA-4K oligomers. The morpholino oligomer also showed some effect in the spleen but was ineffective in thymus tissue. Interestingly, PNA-4K-induced EGFP was also detected in the capsule of the spleen. Overall, the remarkable up-regulation of EGFP indicated that the oligomers distributed to multiple organs of the body, entered the cells and their nuclei and shifted splicing of EGFP-654 pre-mRNA.

Surprisingly, PNA-1K, which in cell culture experiments under conditions of free uptake was more potent than its 2′-O-MOE analogue, showed a total lack of antisense effects in any tissue even after four daily IP injections at 50 mg/kg. These results were confirmed by RT-PCR.

### EXAMPLE 20

Antisense Effects at the mRNA Level.

To confirm that the up-regulation of EGFP signal was indeed due to sequence-specific shift in splicing of EGFP-654 pre-mRNA, total RNA isolated from the tissues was analyzed by RT-PCR. As expected, tissues that showed no fluorescent response to the antisense oligomers (e.g. brain, skin and stomach) also showed no changes in the splicing patterns of EGFP-654. Bone marrow, which was not analyzed by fluorescence microscopy, showed virtually no correction of aberrant splicing in response to antisense treatment. Due to degradation of RNA by pancreatic ribonuclease, tissue from the pancreas was not detectable by RT-OCR. Tissues such as liver and small intestine that showed bright fluorescence also showed a robust increase in the ratio of correctly to aberrantly spliced EGFP-654 mRNA. The PNA-1K oligomer produced no increases in correctly spliced EGFP-654 mRNA.

Quantitation of the RT-PCR results agreed with EGFP fluorescence-based data. For example PNA-4K was more effective than the other two compounds in all tissues but small intestine. In the small intestine, the most effective oligomer was the 2′-O-MOE. After four days of treatment, PNA-4K elicited approximately 40% shifts in splicing in the kidney and liver while responses to 2′-O-MOE and morpholino were in the 20-30% range. Similar ratios were seen in the lung and muscle.

Sequence-specificity of the in vivo effects of antisense oligonucleotides was determined by using 2′-O-MOE, morpholino or PNA-4K control oligomers containing three mismatches to the target sequence. After IP injection of mice with four daily doses of 50 mg/kg, there was little or no correction of aberrant splicing as shown by RT-PCR of total RNA of the treated tissues. In particular, in tissues such as liver and small intestine where oligomers having no mismatches were active, there was virtually no splicing correction after treatment with control oligomers of any backbone.

### EXAMPLE 21

Generation of EGFP Transgenes.

The chicken beta-actin (CX) EGFP plasmid containing no intron was obtained from Masaru Okabe at Osaka University, Japan. The mutant 654 or 705U β-globin IVS2 intron was amplified by PCR from separate plasmids with primers that partially overlapped the coding sequence of EGFP at the area of insertion. The CX-EGFP plasmid was linearized at position 105 of the coding sequence, and both pieces of DNA were used to transform Max DH5X cells. The resulting plasmids were designated CX-EGFP-654 and CX-EGFP-705U. For the generation of the CX-EGFP plasmid with the wild-type β-globin IVS2 intron, unique restriction sites in both the CX-EGFP-654 and CMV-EGFP-WT plasmids were determined at points on either side of the 654 point mutation within IVS2. Both the wild-type intron insert and the CX-EGFP-654 plasmid lacking the insert were used to transform bacteria cells as described above. The resulting plasmid was designated CX-EGFP-WT. For all transgenes, unique restriction sites were used to excise the gene from the plasmid.

### EXAMPLE 21

Transgenic Mice.

Pre-pubescent females were superovulated by IP injection with Pregnant Mares Serum gonadotropin (PMSg). Forty-eight hours later, they were injected with Human Chorionic gonadotropin (HCG) and mated with males for one night. The mice were then removed the following morning for harvesting of pre-implantation embryos. Using a microinjection needle, the DNA solution containing the dsDNA transgene was inserted into the pronucleus of a pre-implanted embryo. The microinjected embryo was then cultured overnight in an incubator. Females, 6-8 weeks of age, were mated to vasectomized males. 0.5 days after mating, the females were anesthetized with avertin and embryos were transplanted into the oviduct. The females were monitored daily until the transferred embryos were born and weaned. Weanlings containing the uniquely altered DNA code (founders) were mated to either a male or female from the background strain. Females, either one or two at a time, were housed with male mice. Litters born in the cages were removed at weaning and separated by sex. The transgenic and/or wild type pups were set aside for later use in specific experiments.

### EXAMPLE 22

Genotyping.

Detection of the transgene in the mice was performed by real-time PCR of genomic DNA isolated from a tail clipping of each animal. Specifically, tail clips were digested in proteinase K overnight at 55° C. in 200 μL total volume. For PCR, 1 μL was used in a reaction containing a forward (^{5′}AGCAAAGACCCCAACGAGAA^{3′}) SEQ ID No. 21 and reverse primer (5′ TCCCGGCGGCGGTCACGAA) SEQ ID No. 22 as well as a double-labeled probe (^{5′}6FAM-CGCGATCACATGGTCCTGCTGG-TAMRA^{3′}) SEQ ID No. 23 for 40 cycles. Real-time PCR was performed on a Perkin-Elmer ABI PRISM 7700 Sequence Detection System.

### EXAMPLE 23

Treatment of EGFP Animals with Oligonucleotides.

Transgenic mice were injected with a 200 μL solution of the indicated concentrations of oligonucleotide in phosphate buffered saline (PBS) by intraperitoneal injection. One injection was given at the same time each day for the indicated number of days. The day after the last injection, mice were fixated by carbon dioxide and organs were removed. A portion of each tissue was cut into small pieces (<2 mm thick) and fixed in 2 mL of 4% paraformaldehyde in PBS. The remainder of each organ was snap frozen in liquid nitrogen.

### EXAMPLE 24

Frozen Tissue Sections.

The fixed tissue slices were removed from the paraformaldehyde and blotted briefly to remove excess fluid. The tissues were then placed in cryomolds and immersed in O.C.T. mounting medium (Miles Scientific, Naperville, Ill.). The molds were frozen slowly to allow for extrusion of any air bubbles from the O.C.T. A cryostat was used to cut 10 mm frozen sections, which were then thaw-mounted onto glass slides and kept at −20 degrees C. or cooler. Images of each slide were taken with a Zeiss fluorescence microscope. Images were digitized with Scion Image software.

### EXAMPLE 25

Isolation of Total RNA and RT-PCR.

Approximately 25 mg of each snap frozen tissue or the samples of cultured cells were homogenized in the presence of 1 mL of TRI-Reagent (MRC, Cincinnati, Ohio). After sufficient agitation, the samples were centrifuged for 2 minutes to remove any undissolved cellular debris, and the supernatant was transferred into a new tube. RNA isolation was carried out according to the manufacturer. 200 ng of total RNA was used in RT-PCR with rTth enzyme (Perkin-Elmer, Branchburg, N.J.) in the presence of 0.2 μCi of α-[³²P]dATP according to the manufacturer's protocols. The reverse transcription reaction was carried out at 70° C. for 15 minutes followed by PCR: 1 cycle, 95° C., 3 minutes; 18 cycles, 95° C., 1 minute; 65° C., 1 minute. For EGFP mRNA amplification forward and reverse primers were ^{5′}CGTAAACGGCCACAAGTTCAGCG^{3′} SEQ ID No. 24 and ^{5′}GTGGTGCAGATGAACTTCAGGGTC^{3′} SEQ ID No. 25, respectively. The latter primer was used in the reverse transcription step. The PCR products were analyzed by electrophoresis on an 8% non-denaturing polyacrylamide gel. Gels were dried and autoradiographed with Kodak Biomax film at −80° C. Images were digitized by scanning with a Hewlett Packard scanner using Adobe Photoshop software.

### EXAMPLE 25

Hepatocyte and Fibroblast Cultures.

For hepatocytes, the liver of EGFP-654 mice was perfused with a perfusion buffer of RPMI media with 0.53 mg/mL of collagenase (Worthington Type 1, code CLS). After perfusion the cell suspension was placed in a stop solution of RPMI with 10% FBS and 0.5% penicillin/streptomycin. Cells were then centrifuged and resuspended in a seeding solution of stop solution plus 1 nM insulin and 13 nM dexamethasone. Approximately 3×10⁵ cells were seeded on a 6-well collagen coated plate. One hour later, the seeding media was replaced with maintenance media consisting of seeding media without the 10% FBS. Cells were treated 24 hours later with maintenance media containing varying levels of oligonucleotide/lipid complexes. Fluorescence images were taken with an Olympus microscope and images were captured using Scion Image software. For Fibroblasts, tail clippings were cut into small pieces and digested in a PBS solution containing 0.125% trypsin, and 1.2 U/mL of dispase. The solution was rotated for 15 minutes at 37° C. for 15 minutes. The supernatant containing cells was transferred to a new tube containing DMEM/F-12 media with 20% FBS. The trypsin/dispase solution was reapplied to the tailpieces and incubated. After 3 cycles, the tailpieces were discarded and the cells in the suspension was counted and seeded in 24 well plates at 1×10⁵ cells/well. Approximately 24 hours later, varying amounts of oligonucleotide/lipid complexes were applied. After another 24 hours, the transfection of both the hepatocytes and the fibroblasts was halted by lysing with TRI-Reagent.

What is claimed:

1. A method of modulating in vivo uptake of a peptide nucleic acid compound comprising modifying said peptide nucleic acid molecule with a positively charged conjugate, said positively charged conjugate having at least three positively charged amino acid units.

2. The method of claim 1 wherein said positively charged conjugate includes at least three lysine, histidine, ornithine or arginine amino acid units.

3. The method of claim 1 wherein said positively charged conjugate includes at least four lysine, hisitidine, ornithine or arginine amino acid units.

4. The method of claim 2 wherein said positively charged conjugate includes at least four lysine, hisidine, ornithine or arginine amino acid units.

5. The method of claim 4 wherein said positively charged conjugate includes at least four lysine amino acid units.

6. The method of claim 4 wherein said positively charged conjugate includes at least four hisidine amino acid units.

7. The method of claim 4 wherein said positively charged conjugate includes at least four ornithine amino acid units.

8. The method of claim 4 wherein said positively charged conjugate includes at least four arginine amino acid units.

9. The method of claim 4 wherein said positively charged conjugate includes at least four amino acid units and at least one of said four amino acid unites is different than a further of said amino acid units.

10. A method of modulating tissue distribution of a peptide nucleic acid compound comprising modifying said peptide nucleic acid molecule with a positively charged conjugate, said positively charged peptide conjugate having at least three positively charged amino acid units.

11. A method of increasing cellular uptake of a peptide nucleic acid compound comprising modifying said peptide nucleic acid molecule with a positively charged conjugate, said positively charged peptide conjugate having at least three positively charged amino acid units.

12. A method of modulating uptake or tissue distribution of a peptide nucleic acid compound in an animal comprising modifying said peptide nucleic acid molecule with a positively charged conjugate, said positively charged conjugate having at least three positively charged amino acid units and delivering said modified peptide nucleic acid compound to said animal.

13. A peptide nucleic acid conjugate of the formula: 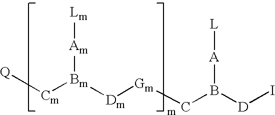
wherein:
m is an integer from 1 to about 50;
L and Lₘ independently are R¹²(R¹³)ₐ; wherein:
R¹² is hydrogen, hydroxy, (C₁-C₄)alkanoyl, a naturally occurring nucleobase, a non-naturally occurring nucleobase, an aromatic moiety, a DNA intercalator, a nucleobase-binding group, a heterocyclic moiety, a reporter ligand, a conjugate or a cationic conjugate;
provided that at least one of R¹² is a naturally occurring nucleobase, a non-naturally occurring nucleobase, a DNA intercalator, or a nucleobase-binding group;
R¹³ is a conjugate; and
a is 0 or 1;
C and Cₘ independently are (CR⁶R⁷)y; wherein:
R⁶ and R⁷ independently are hydrogen, a side chain of a naturally occurring alpha amino acid, (C₂-C₆) alkyl, aryl, aralkyl, heteroaryl, hydroxy, (C₁-C₆) alkoxy, (C₁-C₆) alkylthio, a conjugate, a cationic conjugate, NR³R⁴, SR⁵ or R⁶ and R⁷ taken together complete an alicyclic or heterocyclic system;
wherein R⁵ is hydrogen, a conjugate, or a cationic conjugate,(C₁-C₆)alkyl, hydroxy-, alkoxy-, or alkylthio-substituted (C₁-C₆)alkyl; and
R³ and R⁴ independently are hydrogen, a conjugate, or a cationic conjugate, (C₁-C₄)alkyl, hydroxy- or alkoxy- or alkylthio-substituted (C₁-C₄)alkyl, hydroxy, alkoxy, alkylthio or amino;
D and Dₘ independently are (CR⁶R⁷)_{z};
each of y and z is zero or an integer from 1 b 10, wherein the sum y+z is greater than 2 but not more than 10;
Gₘ is independently —NR³CO—, —NR³CS—, —NR³SO—, or —NR³SO₂— in either orientation;
each pair of A-Aₘ and B-Bₘ are selected such that:
(a) A or Aₘ is a group of formula (IIa), (IIb) or (IIc) and B or Bₘ is N or R³N⁺; or
(b) A or Aₘ is a group of formula (IId) and B or Bₘ is CH; 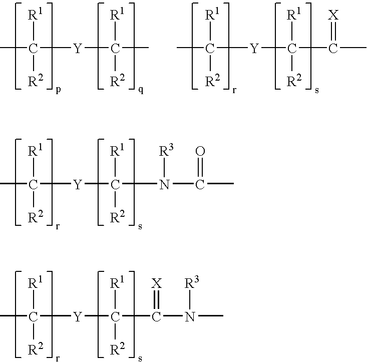
wherein:
X is O, S, Se, NR³, CH₂ or C(CH₃)₂;
Y is a single bond, O, S or NR⁴;
each of p and q is zero or an integer from 1 to 5;
each of r and s is zero or an integer from 1 to 5;
R¹ and R² independently are hydrogen, (C₁-C₄)alkyl, hydroxy-substituted (C₁-C₄)alkyl, alkoxy-substituted (C₁-C₄)alkyl, alkylthio-substituted (C₁-C₄)alkyl, hydroxy, alkoxy, alkylthio, amino, or halogen;
I is —NR⁸R⁹ or —NR¹⁰C(O)R¹¹; wherein:
R⁸, R⁹, R¹⁰ and R¹¹ independently are hydrogen, alkyl, an amino protecting group, a reporter ligand, an intercalator, a chelator, a peptide, a protein, a carbohydrate, a lipid, a steroid, a nucleoside, a nucleotide, a nucleotide diphosphate, a nucleotide triphosphate, an oligonucleotide, an oligonucleoside, a soluble polymer, a non-soluble polymer, a conjugate or a cationic conjugate;
Q is —CO₂H, —CO₂R⁸, —CO₂R⁹, —CONR⁸R⁹, —SO₃H, —SO₂NR¹⁰R¹¹ or an activated derivative of —CO₂H or —SO₃H;
wherein at least one of said R³, R⁴, R⁵, R⁶, R⁷, R⁸, R⁹, R¹⁰, R¹¹, R¹² is said cationic conjugate;
wherein said cationic conjugate includes at least three positively charged amino acid units; and
wherein said cationic conjugate optionally includes a linking moiety.

14. A peptide nucleic acid conjugate of claim 13 wherein said conjugate includes a linking moiety.

15. A peptide nucleic acid conjugate of claim 13 wherein at least one group R⁸ is a cationic conjugate.

16. A peptide nucleic acid conjugate of claim 13 wherein at least one group R⁹ is a cationic conjugate.

17. A peptide nucleic acid conjugate of claim 13 wherein at least one of R¹⁰ is a cationic conjugate.

18. A peptide nucleic acid conjugate of claim 13 wherein at least one of said R¹¹ is a cationic conjugate.

19. A peptide nucleic acid conjugate of claim 17 wherein at least one of said B-Bₘ groups or said G-Gₘ groups include at least one group R³.

20. A peptide nucleic acid conjugate of claim 13 wherein at least one of R⁸, R⁹, R¹⁰ and R¹¹ is a cationic conjugate and said cationic conjugate includes at least four amino acid units.

21. A peptide nucleic acid conjugate of claim 13 wherein at least one of said groups Q or I include at least one of groups R⁸, R⁹, R¹⁰ and R¹¹, and wherein at least one of R⁸, R⁹, R¹⁰, and R¹¹ is a cationic conjugate comprising at least three lysine, hisidine, omithine or arginine monomers.

22. A peptide nucleic acid conjugate of claim 21 wherein said cationic conjugate comprises at least four lysine monomers.

23. A peptide nucleic acid conjugate of claim 13 wherein at least one of R³ R⁴, R⁵, R⁶ and R⁷ is a conjugate.

24. A peptide nucleic acid conjugate of claim 23 wherein at least one of said groups D-Dₘ, or C-Cₘ include at least one of R³, R⁴, R⁵, R⁶ and R⁷.

25. A peptide nucleic acid conjugate of claim 13 wherein m is from 1 to about 200.

26. A peptide nucleic acid conjugate of claim 1 wherein m is from 1 to about 50.
